(19) United States
(12) Patent Application Publication
(10) Pub. No.: US 2009/0169490 A1
(43) Pub. Date: Jul. 2, 2009

(54) COMPOSITION AND METHOD FOR WEIGHT LOSS

(75) Inventors: Jinkui Xie, Guangzhou (CN); Minhua Liu, Guangzhou (CN)

Correspondence Address: SCIHEAD INTELLECTUAL PROPERTY LAW GROUP, LTD, Huihua Commercial & Trade Building, Suite 1508, 80 Xian Lie Zhong Road, Guangzhou, 51070, CN

(73) Assignee: Guangzhou Konzern Pharmaceutical Co., Ltd

(21) Appl. No.: 12/343,437

(22) Filed: Dec. 23, 2008

(30) Foreign Application Priority Data
Dec. 26, 2007 (CN) 200710032895.7

(51) Int. Cl. A61K 9/68; A61K 31/19; A61P 3/04

(52) U.S. Cl. 424/48; 514/574

(57) ABSTRACT

A weight loss composition consisting of 1 to 10 weight parts of L-hydroxy citric acid and 1 to 10 weight parts of L-carnitine tartrate as active components, and pharmaceutically or food-acceptable carrier is disclosed. The present composition can be made to tablet, capsule, oral juice or chewing gum which shows great effect of weight loss.

### FIELD OF THE INVENTION

The present invention relates to a certain composition for weight loss. In another aspect, the invention is directed to a weight loss method, comprising administering to a subject in need of weight loss an effective amount of the composition.

### BACKGROUND OF THE INVENTION

Obesity has reached epidemic proportions globally, with more than 1.2 billion adults overweight—at least 300 million of them clinically obese—and is a major contributor to the global burden of chronic disease and disability. Obesity and being overweight pose a major risk for serious diet-related chronic diseases, including type 2 diabetes, cardiovascular disease, hypertension and stroke, and certain forms of cancer. The health consequences range from increased risk of premature death, to serious chronic conditions that reduce the overall quality of life. Thus, a great of efforts have been made on the research of scientific prevention and treatment of obesity.

Conventionally commercially available weight loss drug are generally classified as follows:

(1) Central inhibition type(appetite suppressants), for example, drugs which effect on the catecholamines metabolic pathway, such as benzene alanines (eg. dextroamphetamine), chlorphentermine, phentermine and amfepramone; drugs which effect on 5-hydroxy tryptamine metabolic pathway, such as fenfluramine and laevofenfluramine, etc; and drugs which effect on both pathways, such as sibutramine. These drugs have almost the same poison side effects, and can neither be used for person suffering from coronary artery disease(CAD), congestive heart failure disease, arrhythmia, stroke or severe hepatic and renal damage, nor person who cannot control his blood pressure or has the history of high blood pressure disease, narrow cleft glaucoma or epilepsy;

(2) Digestion and uptake blockers, such as orlistat with trade name of Xenicale®. It also results in many side effects, such as oily spotting, gas with discharge, fecal urgency, fatty/oily stools, frequent bowel movements and lipid soluble vitamin deficiency, and the like. The drug can neither be used for person suffering from chronic malabsorption syndrome or cholestasis, nor person who is hypersusceptible to any components incorporated in the drug or other formulations;

(3) Metabolism stimulant, for example, \textcircled{1} central stimulants, such as mixtures of ephedrine and caffeine, and \textcircled{2} hormones, such as thyroid harmone, chorionic gonadotrophin, growth harmone and adiposine. They have side effect such as increase in heart rate and myocardial oxygen consumption, inducing to angina pectoris, tension and hyperhidrosis, and so on. These drugs are forbidden to be used on person suffering from hypertension, cardiovascular and cerebrovascular diseases or endocrinous diseases such as hyperthyroidism.

As described above, the existing weight loss products have disadvantages of various side effects, costliness and easy to come back. It is desirable to provide a new weight loss product which is of high efficiency, cost effective, relatively safe and free of side effects.

### SUMMARY OF THE INVENTION

An object of the present invention is to provide a weight loss composition consists of 1 to 10 weight parts of L-hydroxy citric acid and 1 to 10 weight parts of L-carnitine tartrate as active components, and pharmaceutically or food-acceptable carrier.

In one embodiment of the present invention, the ratio of the weight of L-hydroxy citric acid to that of L-carnitine tartrate is 1:1. In another embodiment of the present invention, the L-hydroxy citric acid derived from Garcinia Cambogia. In various embodiments of the present invention, the present composition can be made to tablet, capsule, oral juice or chewing gum.

The present invention omits the asiaticosides described in the application no. 200610036736.X, filed on Jul. 28, 2006. The active components of the present composition only consist of L-hydroxy citric acid and L-carnitine tartrate. As confirmed by experiments, it is surprise to find that the weight loss product of the present invention has a very high efficiency of weight loss. Also, the price of the commercial asiaticosides is extremely high (about 335 USD/1 kg) and thus the price of compositions comprising this component will be correspondingly high. Therefore, few people can afford it and thus will limit the distribution of the product. The present composition does not comprise asiaticosides, and as proved by the experiments in the embodiments below, remains significant effect of weight loss. The compositions of the present invention are of high efficiency, cost effective, relatively safe and free of side effects and most importantly can be widely used by people of different consumption level.

The present invention also provides a method to treat obesity comprising administration of an effective amount of the present weight loss composition to a subject in need thereof.

Definitions

1. L-hydroxy citric acid

Term “L-hydroxy citric acid (HCA)” used in the present invention has a molecular formula of C₆H₈O₈ and molecular weight of 208.12 Dalton. It has a remarkable weight loss effect and possesses the functions of:

(1) inhibiting citric acid converting to acetyl coenzyme A by suppress the activity of the ATP-citric acid catenase, so as to prevent the carbohydrate from converting to fat and thus reduce the synthesis of fatty acid, cholesterol and low density protein;

(2) allowing for the receptor factor in the liver sending signals of “full feeling” to the brain by facilitate the formation and accumulation of muscleglycogen and liver glycogen, thereby reducing appetite. HCA derived from Garcinia Cambogia reduce appetite whilst without side effects such as insomnia, tiredness, weakness and tension;

(3) accelerating consumption of fat and providing energy for body by accelerating the process of oxidation of short chain fatty acids in cytochylema.

The L-hydroxy citric acid also has the functions of improving health condition of cardiovascular system, reducing cholesterol and triglyceride; allowing athletes to obtain muscle without increase in fat, thereby increasing energy; allowing insulin in the body of diabetics to function more effectively; helping to stabilize glucose level in blood; and controlling occurrence of hypoglycemia.

2. L-carnitine tartrate

L-carnitine (also known as Vitamin BT), has a molecular formula of C₇H₁₅NO₃, and a molecular weight of 161.2 Dalton. It is easy to dissolve in water and has an in vivo half-life of 8.4 hours. It is a very unique amino acid derivative which is widely existed in tissues and is necessary for long chain fatty acid metabolism for energy production. However, because the monomer of L-carnitine is extremely unstable, the present invention uses an relative stable form, that is L-carnitine tartrate, which is able to improve over-weight by:

(1) accelerating β-oxidation of fatty acid and producing energy (ATP) by transferring long chain fatty acid from the outside of the membrane of the mitochondria to the inside through carrier in the form of acylcarnitine;

(2) permeating short chain acyl-coenzyme A through cell membrane into liver where the coenzyme is oxidized, or into kidney where the coenzyme is discharged, thereby preventing an excess accumulation of acyl-coenzyme A in cell organelles which damages the cell;

(3) serving as a low energy organic compound and reacting with acetyl CoA to form acetyl carnitine, which will transfer excess lactic acid out of cells and thus avoids acid poisoning in the cells;

(4) improving availability of carbohydrates and amino acids;

(5) providing energy for body by the resultant acylated carnitine stored as a high metabolism energy source in muscle tissues.

Therefore, L-carnitine tartrate has the functions of weight loss, avoidance of fatty liver, fatigue resistivity and anti-aging, etc.

### DETAILED DESCRIPTION OF PREFERRED EMBODIMENTS

The present invention will be detailed described in conjunction of the experiment data provided below.

A variety of weight loss composition can be prepared by mixing L-hydroxy citric acid with L-carnitine tartrate in a suitable proportion and adding a suitable amount of excipient using conventional manufacturing method for food or drug preparation well known in the art.

### EXAMPLE 1

### Tablet Preparation

1000 tablets are prepared by the amounts of components listed in table 1 using conventional method, with each tablet weighing 1 g.

TABLE 1
Components in each 1 kg tablets

| COMPONENTS | WEIGHT (g) |
|---|---|
| L-hydroxy citric acid | 110 |
| L-carnitine tartrate | 180 |
| wheat tarch | 500 |
| hydroxypropyl cellulose | 30 |
| lactose | 150 |
| magnesium stearate | 30 |

### EXAMPLE 2

### Capsule Preparation

Capsule is prepared by mixing 110 g of L-hydroxy citric acid and 110 g of L-carnitine tartrate, with addition of a suitable amount of auxiliary materials, using conventional process well known in the art including agitating, granulating and capsuling.

### EXAMPLE 3

### Oral Juice

Oral juice is made from 50 g of L-hydroxy citric acid and 500 g L-carnitine tartrate, with addition of a suitable amount of flavoring agent such as lecithin and ion exchange resin, and preservative such as benzoic acid and sorbic acid, using conventional process well known in the art.

### EXAMPLE 4

### Chewing Gum

Chewing gum is made from 300 g of L-hydroxy citric acid and 30 g L-carnitine tartrate, with addition of a suitable amount of gum base, flavoring agent and essence, using conventional process well known in the art including agitating, pressing, molding and polishing.

The weight loss products provided by the present invention utilize the synergistic effect and complementary action of these two active components (L-hydroxy citric acid and L-carnitine tartrate). The selling price of the products can be reduce significantly due to the omitting of asiaticosides.

### Laboratory Experiments

I . Experimental Animals:

Male weaned rats (each weighs 50 g) divided into 5 groups with each group 10 rats.

II. Experiment Groups:

The rats are orally administrated L-carnitine tartrate (Group A), L-hydroxy citric acid (Group B), L-carnitine tartrate and L-hydroxy citric acid (Group C), L-carnitine tartrate and L-hydroxy citric acid and asiaticosides (Group D), respectively. For comparison, a control group E is also set herewith. The oral administration dosage is 5 times that for human (kg/day). The rats are fed with feed comprising one of the active components set forth above for continuous 30 days.

III. Operating Steps:

1. Establishment of Diet-Induced Obesity Rat Model

Feed preparation: the feed comprises (a) basic feed consisting of 20 wt % of barley meal, 10 wt % of dehydrated vegetables, 20 wt % of pulse flour, 1 wt % of yeast, 5 wt % of bone dust, 16 wt % of cornmeal, 16 wt % of wheat skin, 10 wt % of fish meal and 2 wt % of salt, and (b) nutritive feed selected from the group consisting of powdered milk, lard, egg and soybean sprout. 10 g of powdered milk, 10 g of lard, one egg and 250 g of soybean sprout are added per 100 g of basic feed.

In the first and second week, each rat is fed 13 g of feed per day, with an increase in the amount of the feed of 2 g every week until the sixth week (23 g per day). The rats are fed twice a day. After the rats are fed with the high-fat, high-nutrition feed above for 45 days, the weight of the rat fed with such feed is nearly two times that of the rat in the control group fed with normal feed (the difference may be more than 100 g).

2. Weight Loss Experiments

After the establishment of the rat model, rats in different groups are orally administrated L-carnitine tartrate (Group A), L-hydroxy citric acid (Group B), L-carnitine tartrate and L-hydroxy citric acid (Group C), L-carnitine tartrate and L-hydroxy citric acid and asiaticosides (Group D). The components are blended in the feed. No additional component is added to the normal feed fed the control group. The variation in weight and percentage body fat during 30 days are observed and reported in table 2.

3. Observations

TABLE 2
Variation in weight and percentage body fat (determined as fat
pad around testicle and kidney).

| | | | | | average percentage body | | |
|---|---|---|---|---|---|---|---|
| | | average weight variation (g) | | | fat variation (g) | | |
| groups | rat No. | before | after | variation | before | after | variation |
| A | 10 | 103.28 | 98.28 | −5 | 6.23 | 3.93 | −2.3 |
| B | 10 | 103.12 | 97.32 | −5.8 | 6.21 | 3.81 | −2.4 |
| C | 10 | 103.10 | 96.6 | −6.5 | 6.22 | 3.42 | −2.8 |
| D | 10 | 103.12 | 96.61 | −6.51 | 6.23 | 3.63 | −2.6 |
| E (control) | 10 | 103.18 | 105.68 | +2.5 | 6.21 | 7.41 | +1.2 |

4. Experiment Data and Results

The experiment data is analyzed by software SPSS12.0 edition, and the result are shown as below:

\textcircled{1} The difference between group A, B, C or D and group E respectively is significant (p<0.01) which shows that the components fed to A, B,C and D has weight loss effect;

\textcircled{2} The difference between group A, or B and group C is significant (p<0.0 1) which shows that L-carnitine tartrate plus L-hydroxy citric acid (group C) has greater effect of weight loss than L-carnitine tartrate (group A) or L-hydroxy citric acid (group B);

\textcircled{3} The difference between group C and group D is not significant (p>0.05) which indicates that L-carnitine tartrate+L-hydroxy citric acid (group C) shows almost the same effect of weight loss as that of L-carnitine tartrate+L-hydroxy citric acid+asiaticosides (Group D).

The weight loss product provided by the present invention only includes L-carnitine tartrate and L-hydroxy citric acid as the active components, and does not contain asiaticosides, while has almost the same weight loss effect as, or even greater effect than that of the compositions containing asiaticosides. The production cost is therefore reduced, and the selling price can be lowered, which makes the product affordable by more people.

What is claimed is:

1. A weight loss composition consisting of 1 to 10 weight parts of L-hydroxy citric acid and 1 to 10 weight parts of L-carnitine tartrate as active components, and pharmaceutically or food-acceptable carrier.

2. The weight loss composition of claim 1, wherein the the ratio of the weight of L-hydroxy citric acid to that of L-carnitine tartrate is 1:1.

3. The weight loss composition of claim 1, wherein the L-hydroxy citric acid derived from Garcinia Cambogia.

4. The weight loss composition of claim 1, wherein the composition is made into tablet.

5. The weight loss composition of claim 1, wherein the composition is made into capsule.

6. The weight loss composition of claim 1, wherein the composition is made into oral juice.

7. The weight loss composition of claim 1, wherein the composition is made into chewing gum.

8. A method for treating obesity comprising administration to a subject in need of weight loss of an effective amount of the weight loss composition in claim 1.
